(19) United States
(12) Patent Application Publication
(10) Pub. No.: US 2005/0152866 A1
(43) Pub. Date: Jul. 14, 2005

(54) Manufacture of alkyl benzene sulphonic acids

(75) Inventors: Hendrick Petrus Frick, Ayr (SC); Ajit Kumar, West Bromwich (GB); Lean-Luc Yves Leon Urbain, Salmagne (FR); Didier Hauss, Verdun (FR); Pierre Solastiouk, Verdun (FR)

Correspondence Address: Huntsman;Legal Department, P O Box 15730, Austin, TX, 78761, US

(73) Assignee: Huntsman International LLC, Salt Lake City, UT (US)

(21) Appl. No.: 10/492,389

(22) PCT Filed: Sep. 25, 2002
(86) PCT No.: PCT/EP02/10738

(30) Foreign Application Priority Data
Oct. 10, 2001 (GB) 0124274.2

(51) Int. Cl. A61K 7/075; A61K 31/185

(52) U.S. Cl. 424/70.24; 514/553

(57) ABSTRACT

A novel, storage-stable, free-flowing, solid product consisting essentially of an alkyl benzene sulphonic acid having 1 to 4 aliphatic carbon atoms and from 7 to 11% water based on the weight of the product, said alkyl benzene sulphonic acid comprising at least 90% by weight of a single isomer based on the total weight of organic material in the product, is prepared by drying a storage-unstable, solid alkyl benzene sulphonic acid having more than 11% by weight water, said drying being effected at sub atmospheric pressure and at a temperature below the softening point of the product.

Lower alkyl benzene sulphonic acids comprising from 1 to 4 aliphatic carbon atoms for use as catalysts or hydrotropes are manufactured by sulphonation of the corresponding alkylbenzenes. In some instances, most notably in sulphonating toluene the sulphonation can provide a solution which precipitates crystals of a single alkyl benzene sulphonic acid in high purity when concentrated and cooled.

Crystals comprising p-toluene sulphonic acid consisting of 99% p-isomer by weight of the total organic matter and about 12% of water are manufactured commercially for use as transesterification and polymerisation catalysts, and in fragrances, agrochemical and pharmaceutical formulations and dental cements. Unfortunately after a few days storage, even in dry conditions the crystals agglomerate into an intractable cementitious mass, which is difficult to break up and to handle.

We have now discovered that this problem may be alleviated by drying at relatively low temperatures and pressures sufficiently to reduce the water content to a value between 7 and 11% by weight of the product. The latter can be obtained as a stable free flowing crystalline solid or a friable aggregate, which is readily crushed to provide a stable free-flowing solid.

Our invention provides a storage-stable, free-flowing, solid product consisting essentially of an alkyl benzene sulphonic acid having 1 to 4 aliphatic carbon atoms and from 7 to 11% water based on the weight of the product, said alkyl benzene sulphonic acid comprising at least 90% by weight of a single isomer based on the total weight of organic material in the product.

According to a second embodiment our invention provides a method of manufacture of an alkyl benzene sulphonic acid product as aforesaid which comprises drying a free flowing solid composition consisting essentially of a single isomer of an alkyl benzene sulphonic acid having from 1 to 4 carbon atoms and more than 11% by weight water, said drying being effected at sub atmospheric pressure and at a temperature below the softening point of the product and being sufficient to lower the water content of the product to the range 7 to 11% by weight of the total product.

In the following discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

Preferably the product contains more than 7.5%, more preferably more than 8%, most preferably more than 8.5%, by weight of water, and preferably less than 10.5%, more preferably less than 10%, most preferably less than 9.5%, based on the total weight of the product.

The preferred alkyl benzene sulphonic acid is p-toluene sulphonic acid, which can be crystallised in high yield from the concentrated liquors obtained by sulphonating toluene. In principle, any other lower alkyl benzene sulphonic acid which can be obtained as a single isomer of more than 90% purity can be employed, e.g. xylene sulphonic acid, ethylbenzene sulphonic acid or cumene sulphonic acid.

The solids preferably comprise more than 95%, more preferably more than 98%, most preferably more than 98.5%, and preferably less than 99.9%, more preferably less than 99.7%, most preferably less than 99.5% by weight of a single alkyl benzene sulphonic acid isomer based on the total solids content of the product.

Drying is preferably effected by vacuum or freeze drying. Normal methods of spray, or fluidised bed drying at elevated temperatures do not provide a satisfactory product. Drying must be carried out below the softening point of the product. This normally requires temperatures below 70° C., preferably below 60° C., more preferably below 50° C., most preferably below 30° C.

The invention will be illustrated by the following examples.

A liquor obtained by sulphonating toluene in a glass lined reactor following by stripping of toluene and concentration to 78% by weight solids on a graphite film evaporator, was cooled from 80° C. to 20° C. 250 gm of fine white crystals, consisting of 12% by weight water and the balance consisting essentially of toluene sulphonic acid (99% p-isomer), were recovered by centrifuging. The product was dried under vacuum at 60° C. to 9% moisture and was still readily friable after 12 months storage. An undried product was a hard intractable mass after 2 days.

1) a storage-stable, free-flowing, solid product consisting essentially of an alkyl benzene sulphonic acid having 1 to 4 aliphatic carbon atoms and from 7 to 11% water based on the weight of the product, said alkyl benzene sulphonic acid comprising at least 90% by weight of a single isomer based on the total weight of organic material in the product.

2) A composition according to claim 1 containing from 7.5 to 10.5%, by weight water.

3) A composition according to claim 2 containing from 8 to 10%, by weight water.

4) A composition according claim 1, comprising at least 95% of a single isomer.

5) A composition according to claim 1, wherein said sulphonic acid is p-toluene sulphonic acid.

6) A method of manufacture of a storage-stable, free-flowing, solid, alkyl benzene sulphonic acid product as claimed in any foregoing claim, which comprises drying a storage-unstable, solid composition consisting essentially of a single isomer of an alkyl benzene sulphonic acid having from 1 to 4 carbon atoms and more than 11% by weight water, said drying being effected at sub atmospheric pressure and at a temperature below the softening point of the product and being sufficient to lower the water content of the product to the range of 7 to 11% by weight of the total product.

7) A method according to claim 6, wherein said storage-unstable, solid product has been obtained by crystallisation from aqueous solution.

8) A method according to claim 7, wherein said aqueous solution has been prepared by a process comprising the steps of reacting an alkyl benzene having from 1 to 4 aliphatic carbon atoms with a sulphonating agent to form a solution of alkyl benzene sulphonic acid.
